(19) United States
(12) Patent Application Publication
(10) Pub. No.: US 2025/0362158 A1
(43) Pub. Date: Nov. 27, 2025

(54) OXYGEN SENSOR ELEVATOR ASSEMBLY

(71) Applicant: RAPID MICRO BIOSYSTEMS, INC., Lowell, MA (US)

(72) Inventor: Ethan FRANCO, Boston, MA (US)

(21) Appl. No.: 18/673,716

(22) Filed: May 24, 2024

(51) Int. Cl. G01D 11/22; G01D 11/10

(52) U.S. Cl. CPC G01D 11/22; G01D 11/10

(57) ABSTRACT

Exemplary embodiments provide a new type of sensor elevator assembly for testing the environment within a microbial growth cassette, and techniques for using the assembly. The sensor elevator may support various types of sensors, such as oxygen or carbon dioxide probes or temperature sensors. In some embodiments, the elevator assembly includes a unique float mechanism in the form of a linear slide and spring system that allows the elevator assembly to be controlled very precisely and provides some cushioning in the event that the sensor makes contact with the optical lid. Thus, the impact and sustained forces exerted on the cassette can be reduced or eliminated. This helps to mitigate the risks of fiber optic damage or cassette misalignment due to excess force exerted during sensing.

### BACKGROUND

Sterility testing is a critical process used to determine the absence or presence of viable microorganisms in a product or sample. It is commonly performed on pharmaceuticals, medical devices, and other sterile products to ensure their safety and effectiveness.

Generally speaking, sterility testing may proceed as follows. First, a representative sample of the product or material to be tested may be obtained. A test method may then be selected. Commonly, membrane filtration is the test method applied because of the advantages it provides in terms of sensitivity, versatility, quantification, compatibility, sample recovery, validation, potential for automation, and time efficiency.

In membrane filtration, the sample is filtered through a membrane filter with a defined pore size. The filter retains any microorganisms present in the sample, allowing for their subsequent detection. The membrane filter may be provided on a suitable analysis container, such as the sterility cassettes offered by Rapid Micro Biosystems, Inc. of Lowell, Massachusetts. As the sample is filtered through the membrane filter, any remaining sample fluid that passes through the membrane filter may be drained through a drain port in the base of the cassette.

After filtration, the membrane filter is aseptically transferred to an appropriate culture medium that supports the growth of a wide range of microorganisms. The culture medium can be broth or agar-based, depending on the testing method used. The inoculated culture medium is then incubated under suitable conditions, typically at a temperature between 20-40° C., for a specified period, often ranging from 2 to 14 days. This allows any viable microorganisms to grow and form visible colonies.

In traditional sterility testing, an aerobic environment is usually employed to promote the growth of aerobic microorganisms. However, certain products or samples may contain anaerobic microorganisms that require an oxygen-free environment to grow and be detected accurately.

An anaerobic environment refers to a condition devoid of (or with significantly reduced) oxygen. Anaerobic conditions are created to facilitate the growth and detection of anaerobic microorganisms, which are organisms that can thrive or survive in the absence of oxygen.

To create an anaerobic environment for sterility testing, specialized systems are used. These systems are designed to maintain low oxygen levels and provide a controlled atmosphere conducive to the growth of anaerobic microorganisms. They typically contain catalysts or chemicals that remove or consume oxygen from the chamber.

The sterility testing process within an anaerobic environment involves transferring a sample into the anaerobic chamber and performing the necessary manipulations, such as inoculating the sample onto appropriate culture media. The chamber is then sealed, and the oxygen is purged or reduced to create the desired anaerobic conditions. The samples are then incubated at suitable temperatures to allow anaerobic microorganisms to grow, if present.

By providing an anaerobic environment, sterility testing can effectively detect and quantify anaerobic microorganisms, ensuring the accuracy and completeness of the microbial evaluation of a product or sample.

After the incubation period, the culture media is examined for the presence of microbial growth. The presence of visible colonies indicates a positive result, suggesting the presence of viable microorganisms and thus a failed sterility test. Alternatively, if no visible growth is observed, it indicates a negative result, suggesting the absence of viable microorganisms and a successful sterility test. In the case of positive results, additional tests can be performed to identify the microorganisms present, such as gram staining, biochemical tests, or molecular techniques like polymerase chain reaction (PCR).

The results of the sterility testing, including the test method, sample details, incubation conditions, and results, are documented as part of the testing records.

### BRIEF SUMMARY

In one aspect, an apparatus includes a sensor probe and a sensor elevator configured to move the sensor toward a surface of a lid of a cassette to measure a property of an environment within the cassette, where the sensor elevator includes a float mechanism configured to reduce or eliminate a sustained or impact force of the sensor on the lid of the cassette. The sensor probe may include one or more of an oxygen sensor probe, a temperature sensor probe, or a carbon dioxide sensor probe. The sensor probe may perform one or more of emitting light or receiving a fluorescence signal.

The float mechanism may include a linear slide and one or more springs. In some embodiments, the float mechanism may include foam cushioning.

The sensor elevator may include a pneumatic actuator for moving the sensor probe toward the surface of the lid of the cassette. The sensor elevator assembly may include an adjuster configured to adjust at least one of the horizontal or vertical positioning of the distal member of the probe support with respect to the proximal member of the probe support.

The sensor elevator assembly may include a base affixed to the float mechanism and configured to support the float mechanism at a predetermined location with respect to the cassette. The sensor elevator assembly may include a probe support to which the sensor probe is affixed, the probe support including a proximal member and a distal member. The distal member of the probe support may be T-shaped and include a distal probe support arm, and the proximal member of the probe support may be L-shaped and include a proximal probe support arm configured to interface with and support the distal probe support arm.

In some embodiments, a method of deploying the apparatus may include moving a cassette to a predefined measurement location with respect to the sensor elevator, moving the sensor elevator to place the sensor probe into a predetermined configuration with respect to the cassette, and measuring a property of the environment within the cassette using the sensor probe.

Measuring the property of the environment within the cassette may include measuring an oxygen concentration, a temperature sensor probe, or a carbon dioxide concentration. The method may include transmitting a control signal configured to cause the sensor probe to perform one or more of emitting light or receiving a fluorescence signal.

Moving the sensor elevator may include moving the sensor probe into contact with the cassette so that the float mechanism compresses.

The method may also include attaching a pneumatic line to a pneumatic actuator of the sensor elevator, and transmitting a control signal configured to cause the pneumatic actuator to move a predefined distance.

The method may also include fixing a base to the float mechanism, the base configured to support the float mechanism at a predetermined location with respect to the cassette. The method may also include affixing the sensor probe to the sensor elevator. The sensor probe may be fixed to a probe support that includes a proximal member and a distal member. The distal member of the probe support may be T-shaped and include a distal probe support arm, and the proximal member of the probe support may be L-shaped and include a proximal probe support arm configured to interface with and support the distal probe support arm. The distal member may be assembled on the proximal member by mounting the distal member arm on the proximal member arm and fastening the proximal member arm and distal member arm together.

The method may also include adjusting an adjuster to change at least one of the horizontal or vertical positioning of the distal member of the probe support with respect to the proximal member of the probe support.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary cassette assembly in accordance with one embodiment.

FIG. 2 is a close-up of a portion of a cross-sectional view of the cassette assembly in accordance with one embodiment.

FIG. 3 depicts an assembled oxygen elevator assembly in accordance with one embodiment.

FIG. 4 depicts a cassette deployed on the exemplary oxygen elevator assembly.

FIG. 5 is an exploded view showing the parts of an oxygen elevator assembly in accordance with one embodiment.

FIG. 6 is a flowchart describing a method of using an oxygen elevator assembly in accordance with one embodiment.

### DETAILED DESCRIPTION

In both aerobic and anaerobic sterility testing, it may be important to know whether the oxygen levels in the cassette are suitable for the type of microbial organisms being tested. One technique for determining the oxygen levels involves deploying an oxygen sensor inside the cassette and taking an optical reading of the oxygen sensor through the clear lid of the cassette. One example of an oxygen sensor suitable for use in such techniques is the Oxygen Sensor Spot manufactured by PreSens Precision Sensing GmbH of Regensburg, Germany, although any number of different types of oxygen sensors may be utilized.

Such a sensor is affixed (e.g., with adhesive) to the inside of the optical lid and includes an oxygen sensitive coating. When excited by an input light signal, the coating fluoresces. The fluorescence varies based on the amount of oxygen in the environment. The fluorescence can thus be measured with an optical sensor and translated into a reading for the oxygen level.

To provide the input excitation light and allow for optical measurement of the resulting fluorescence, a fiber optic cable may be placed in close proximity to the lid in alignment with the oxygen sensor. For example, the fiber optic cable may be clamped to a T-shaped bracket in an elevator assembly. The bracket can then be extended down towards the cassette to read the oxygen levels. However, the oxygen sensor elevator must be controlled with relatively high precision. The end of the fiber optic cable must be placed in close proximity to the clear lid of the cassette in order to get an accurate reading. However, if the elevator assembly is lowered too far, the end of the fiber optic cable will contact the lid. In this case, the optical transmitter/receiver may become damaged, or the cassette may be moved so that the oxygen sensor is no longer aligned to the fiber optic cable.

Accordingly, when setting up a sterility testing system, a technician often needs to calibrate the elevator assembly very carefully. This requires time and expertise.

In addition to oxygen sensors, similar problems exist with temperature sensors, carbon dioxide sensors, and other types of measurement devices that require that a probe be moved into close proximity with the cassette. Although exemplary embodiments will be described with reference to oxygen sensors, it is understood that the present invention may be applied with any other suitable type of sensor or device that needs to be moved into place near the cassette without touching it (or touching it with minimal force).

Exemplary embodiments provide a new type of oxygen sensor elevator assembly that mitigates these issues, and techniques for using the assembly. In these embodiments, the elevator assembly includes a unique float mechanism in the form of a linear slide and spring system that allows the elevator assembly to be controlled very precisely and provides some cushioning in the event that the sensor makes contact with the optical lid. Thus, the impact and sustained forces exerted on the cassette can be reduced or eliminated. This helps to mitigate the risks of fiber optic damage or cassette misalignment due to excess force exerted during sensing.

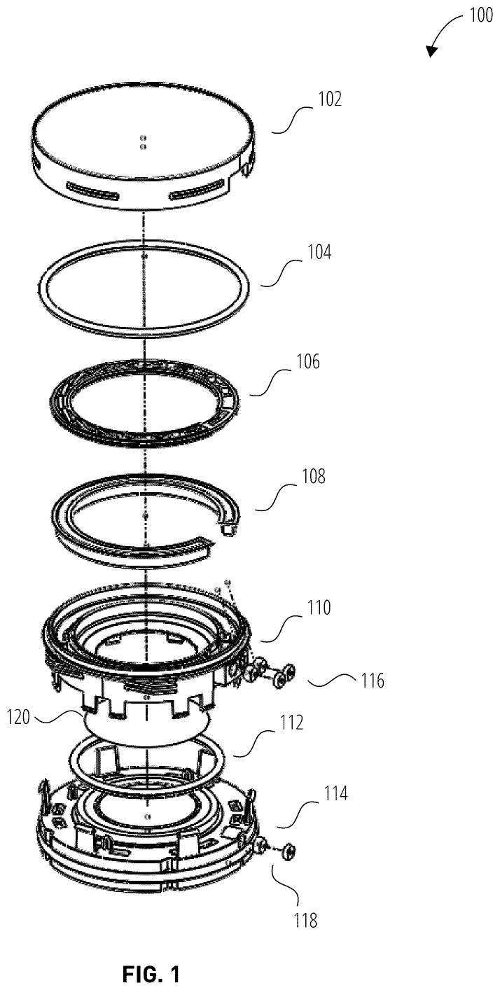

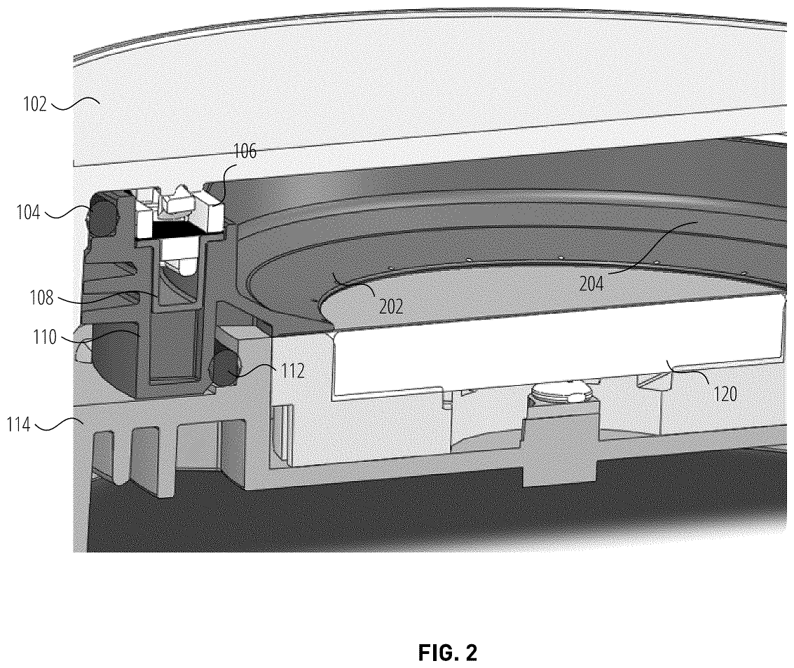

An example of a cassette assembly 100 is shown in FIG. 1, and a cross-sectional side- view is shown in FIG. 2. The cassette assembly 100 may provide a sterile environment for testing. The cassette assembly 100 may provide an anaerobic or aerobic environment, depending on the application. Note that, for ease of discussion, the splashguard described in more detail below is omitted from FIG. 1 and FIG. 2, but can be deployed in the locations noted in connection with subsequent figures.

From top to bottom in FIG. 1, the exemplary cassette assembly 100 includes a lid 102, an o-ring 104, an optional foil cutter 106, a scavenging tray assembly 108, a mid-body assembly 110, a membrane filter 120, a second o-ring 112, and a base assembly 114.

The base assembly 114 forms the bottom-most part of the cassette assembly 100 and serves as a supporting structure to which the other parts can be mounted. The base assembly 114 may be sized and shaped so as to be accommodated in an appropriate testing or analysis device.

A membrane filter 120 may be provided on the base assembly, between the base assembly 114 and the mid-body assembly 110. The membrane filter 120 may be a part of a media pad sized and shaped to be accommodated by a corresponding recess in the base assembly 114. The membrane filter 120 may be any suitable filter, and may have characteristics (such as a desired porosity) selected based on the particular application (e.g., the size of the microorganisms of interest that are intended to be captured by the membrane filter 120). In some embodiments, more than one membrane filter 120 may be provided, which may include multiple different types of membrane filters 120.

Target fluids for analysis may be passed through the membrane filter 120 and into the base assembly 114. The base assembly 114 may include a drain port 118 that allows the fluids to be removed from the cassette assembly 100 after filtration. The drain port 118 may include an opening provided in a part of the base assembly 114 internal to the cassette assembly 100 that connects to a specially shaped outlet on the exterior side of the cassette assembly 100. The outlet may be sized and shaped to mate with a drain manifold that receives the removed fluid and delivers it to an appropriate disposal location.

An o-ring 112 may be provided between the base assembly 114 and the mid-body assembly 110 to prevent fluid from leaking around and therefore bypassing the membrane filter 120. The mid-body assembly 110 includes a mid-body inlet 116 that allows the target fluid (or fluids) being analyzed to be admitted into the cassette assembly 100. The mid-body inlet 116 may include an opening provided in a part of the mid-body assembly 110 internal to the cassette assembly 100 that connects to an opening on the exterior side of the cassette assembly 100. Within the mid-body inlet 116 may be a structure, such as a rubber septum, that seals the cassette assembly 100. To admit a target fluid into the cassette assembly 100, a needle may be used to pierce the structure in the mid-body inlet 116 and deliver the fluid at a relatively high pressure.

In some embodiments, more than one mid-body inlet 116 may be included in the mid-body assembly 110. For example, one mid-body inlet 116 may be provided for admitting a first sample (target fluid of interest for analysis) into the cassette assembly 100, while a second mid-body inlet 116 is provided for admitting a second, different sample. In other embodiments, a first mid-body inlet 116 may be provided for admitting a sample, while a second mid-body inlet 116 may be provided for admitting a growth medium.

The top of the mid-body assembly 110 may be shaped to accommodate a scavenging tray assembly 108, which may include a scavenging material that (for example) absorbs oxygen in the cassette assembly 100. The scavenging tray assembly 108 may be topped by foil that holds the scavenging material in place and protects it from outside air until the scavenging tray assembly 108 is deployed in the cassette assembly 100. To release the scavenging material, the cassette assembly 100 may be provided with a foil cutter 106 designed to penetrate the foil and allow the scavenging material to scavenge the environment within the sealed cassette assembly 100.

To seal the cassette assembly 100, an o-ring 104 may be placed on top of the mid-body assembly 110, and then a lid 102 may be used to cap the entire assembly. As shown in FIG. 2, the o-ring 104 forms a seal between the mid-body assembly 110 and the lid 102 and prevents the fluid from leaking from the top of the cassette assembly 100 (and seals the interior of the cassette assembly 100 to allow the scavenging material to scavenge the environment of oxygen).

As further shown in FIG. 2, the mid-body assembly 110 may include a mid-body assembly floor 202 that extends from an inner circumferential wall 204 of the mid-body assembly 110 towards an interior of the cassette assembly 100 in the radial direction. The mid- body assembly floor 202 may be slanted towards the membrane filter 120 to encourage the fluid to flow towards the membrane filter 120.

Although exemplary embodiments are described with reference to the depicted cassette assembly configuration for purposes of illustration, one of skill in the art will recognize that other types of cassette assemblies (with more, fewer, or a different configuration of parts) or other sterile environments may also be used. Moreover, although exemplary embodiments are described in terms of sterility testing using membrane filtration (and the structure in FIG. 1 and FIG. 2 is configured accordingly), other applications of the splashguard described below will be readily apparent.

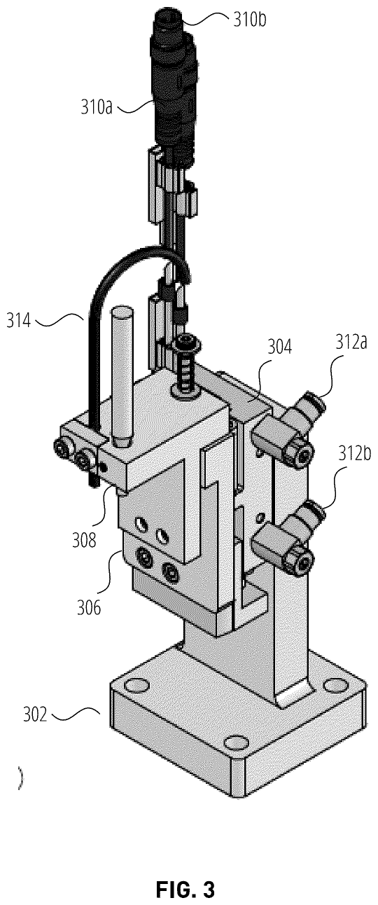

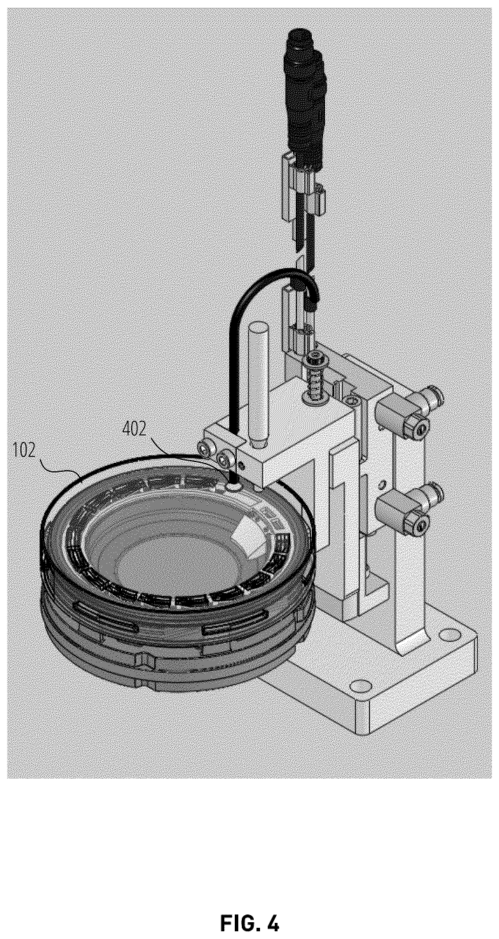

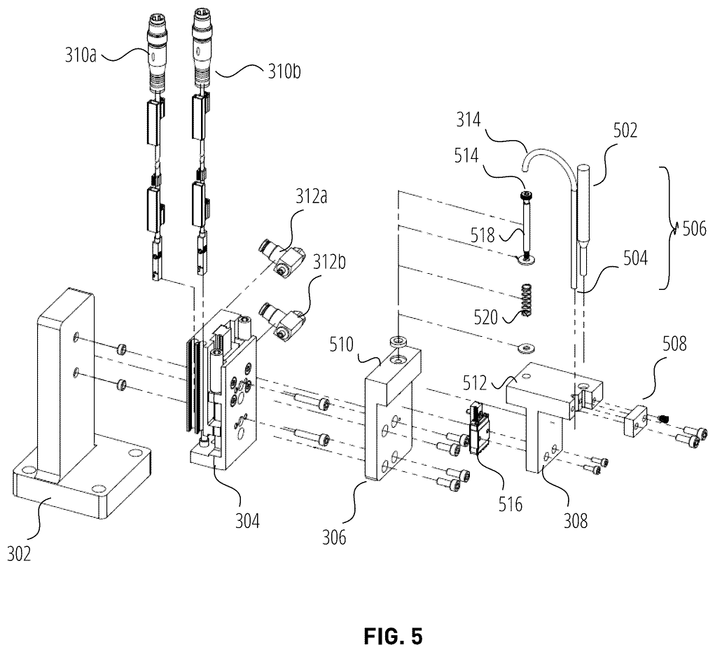

FIGS. 3-5 depict an example of a sensor elevator assembly suitable for use with an aerobic or anaerobic cassette assembly 100. FIG. 3 depicts an assembled sensor elevator in isolation, while FIG. 4 depicts the assembled sensor elevator with a deployed cassette ready for measurement. FIG. 5 depicts an exploded view of the sensor elevator showing the various parts of the sensor elevator. FIGS. 3-5 are discussed together for ease of discussion.

The sensor elevator assembly includes structural elements, including a base 302, a float mechanism 304, a probe support (proximal) 306, and a probe-support (distal) 308.

The base 302 includes an attachment surface (extending horizontally at the bottom of FIGS. 3-5) and a support column (extending vertically in FIGS. 3-5). The attachment surface may be configured to support the other elements of the sensor elevator assembly and may be configured to attach or mate with an imaging deck of an analysis device such as a sterility testing device. The attachment surface may therefore be provided with one or more features configured to mate to a corresponding face of the imaging deck, or may include through-holes for fasteners such as screws so that the base 302 can be secured to the imaging deck. In some embodiments, the base 302 may be integral with the imaging deck.

The float mechanism 304 may be or may include two or more sliding surfaces and an actuator configured to move one of the sliding surfaces towards or away from a cassette assembly deployed in proximity to the sensor elevator (see FIG. 4). For example, the actuator may be a pneumatic or hydraulic actuator, an electrical linear actuator, a rotary actuator connected to the slide through a transmission that converts the rotary motion of the actuator to a linear motion of the sliding surfaces, etc. In the depicted example, the actuator is a pneumatic actuator, and accordingly pneumatic connectors 312a, 312b are provided to allow pneumatic fluid lines to be connected to the actuator. The pneumatic fluid may be supplied to and/or removed from the pneumatic connectors 312a, 312b to cause the actuator to move the sliding surfaces with respect to each other. If another type of actuator is used, suitable connection points for supplying (e.g.) and electric current, hydraulic fluid, etc. may be provided.

The probe support (proximal) 306 may be configured to attach to one of the sliding surfaces of the float mechanism 304. As the sliding surface moves up or down (in this example), the probe support (proximal) 306 may be carried with the sliding surface, thus moving the probe support (proximal) 306 up or down as well.

The probe-support (distal) 308 may be configured to attach to the probe support (proximal) 306 and may support a sensor probe 506 that measures a parameter of an environment within the cassette through the lid 102 of the cassette. This may be achieved, for example, by affixing an oxygen sensor 402 to the inside of the cassette lid 102 as previously described. Other types of sensors may also be used and may be read through the lid 102 of the cassette. The sensor probe 506 may include one or more sensors, such as a temperature sensor 502 and/or an optical transmitter/receiver 504 for an optical sensor.

The optical transmitter/receiver 504 may serve as the terminal end of a fiber optic cable 314. In one embodiment, the fiber optic cable 314 may attach to an electro-optical module that transmits light through the fiber optic cable 314 to the the optical transmitter/receiver 504 to cause the optical transmitter/receiver 504 to emit the light, and processes the resulting fluorescence signal that is received from the optical transmitter/receiver 504.

To secure the sensor probe 506 to the probe-support (distal) 308, a probe securer 508 may optionally be attached to the probe-support (distal) 308. The probe securer 508 and/or probe-support (distal) 308 may have surfaces with indentations or other features that are shaped and configured to mate with the shape and configuration of the sensor probe 506.

The probe support (proximal) 306 and probe-support (distal) 308 may include surfaces configured to interact or mate with each other. For example, the probe support (proximal) 306 may be substantially “L” shaped, with a support column parallel to and aligned with one of the sliding surfaces of the float mechanism 304 and a proximal probe support arm 510 extending substantially perpendicular to the support column. Similarly, the probe-support (distal) 308 may include a support column parallel to and aligned with the support column of the probe support (proximal) 306. The support columns of the probe support (proximal) 306 and probe-support (distal) 308 may be fastened together. In the depicted embodiment, the support columns are fastened together with an intermediate horizontal calibration adjuster 516 disposed in between the two. The horizontal calibration adjuster 516 may be a simple spacer, or may be extendible in the horizontal direction in order to move the support column of the probe-support (distal) 308 towards or away from the support column of the probe support (proximal) 306. In this way, the sensor probe 506 may be movable towards or away from the float mechanism 304, thus allowing the horizontal position of the sensor probe 506 to be adjusted.

Alternatively or in addition, a vertical calibration adjuster 514 may be provided in the form of a screw 518 that tightens against a spring 520, or any other element capable of moving the probe-support (distal) 308 in a vertical direction with respect to the probe support (proximal) 306.

In the depicted example, the vertical calibration adjuster 514 is assembled by providing a first washer in a countersunk and threaded machined hole in the proximal probe support arm 510 of the probe support (proximal) 306. The probe-support (distal) 308 is placed on top of the probe support (proximal) 306 and a corresponding threaded hole in the distal probe support arm 512 of the probe-support (distal) 308 is aligned to the threaded hole in the proximal probe support arm 510. A washer is placed on top of the hole in the distal probe support arm 512, and the spring 520 is placed on top of this second washer. A third washer is placed on top of the spring 520 and the screw 518 is threaded through the third washer, the spring 520, the second washer, the hole in the distal probe support arm 512, the first washer, and finally is screwed into the threaded hole in the proximal probe support arm 510.

As the vertical calibration adjuster 514 is screwed into the distal probe support arm 512 of the probe-support (distal) 308 and thus pushes against the proximal probe support arm 510 of the probe support (proximal) 306, the probe-support (distal) 308 and probe support (proximal) 306 can be pushed away from each other or pulled towards each other (depending on the configuration) in the vertical direction. This allows the height of the probe-support (distal) 308 that carries the sensor probe 506 to be changed. In preferred embodiments, a technician will adjust the maximum extension distance of the sensor probe 506 using the vertical calibration adjuster 514 so that, when the float mechanism 304 is fully actuated and at its lowest (in this example) position, the optical transmitter/receiver 504 of the sensor probe 506 is directly adjacent to (and, in some embodiments, touching) the lid 102 of the cassette assembly 100. In some embodiments, the maximum extension distance is set so that the optical transmitter/receiver 504 and the lid 102 are allowed to come into contact with each other, so that the cushioning elements such as the springs 310a, 310b absorb some of the impact force of the optical transmitter/receiver 504 on the lid 102 and reduce the sustained force while these elements are in contact (as compared to a system with a rigid actuator that omits such a float mechanism 304). For example, in some embodiments the sustained force may be limited to, at most, 0.451 b. In such embodiments, the cushioning elements may be springs having a rate of up to (and including) 2.2 lb/in with a maximum compression up to (and including) 0.2 in.

In some embodiments, any or all of the base 302, float mechanism 304, probe support (proximal) 306, and probe-support (distal) 308 may be made integral with each other. In other embodiments, some of these elements may be omitted. The base 302, probe-support (distal) 308, probe support (proximal) 306, float mechanism 304, and/or probe securer 508 may be made from any suitable material. In one embodiment, these components are made from machined and anodized 6061-T6 aluminum.

The float mechanism 304 may further include one or more cushioning elements, such as spring 310a, 310b or cushioning foam. The cushioning elements may be positioned and configured such that, as one sliding surface of the float mechanism 304 carries a sensor probe 506 towards a lid 102 of the cassette, the cushioning elements begin to be engaged and compressed when the sensor probe 506

Each of the base 302, float mechanism 304, probe support (proximal) 306, and probe-support (distal) 308 may include through-holes or other appropriate features to allow these elements to be secured to each other. FIGS. 3-5 depict structural elements that are secured together with fasteners such as screws, but other attachment elements (such as mating surfaces) may also be used.

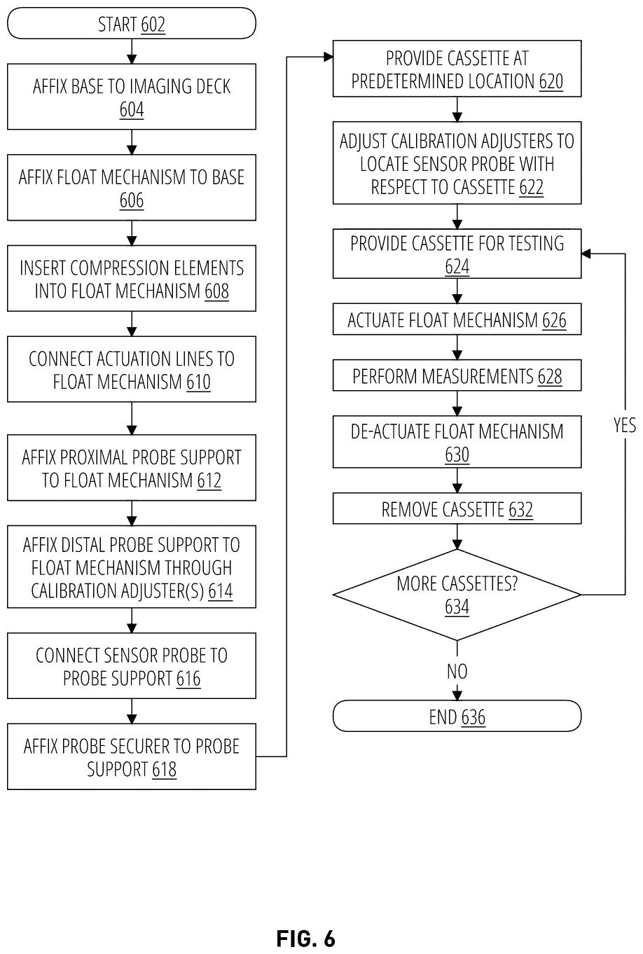

FIG. 6 illustrates an example routine for deploying and using a sensor elevator assembly. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

The method starts at start block 602. At block 604, a base 302 may be provided on or affixed to an imaging deck of a sample analysis device, such as a sterility testing device. The base 302 may be a base as described above in connection with FIGS. 3-5 and may be screwed into the imaging deck through one or more through-holes provided in an attachment surface of the base 302. In embodiments where the base 302 and the imaging deck are integral, it may not be necessary to secure the base 302 to the imaging deck.

At block 606, a float mechanism 304 may be affixed or secured to the base 302. The float mechanism 304 may be a float mechanism as described above in connection with FIGS. 3-5 and may be secured by mating corresponding surfaces in the float mechanism 304 and base 302 together and/or by fastening the two elements together by passing fasteners through through-holes in the float mechanism 304 and attaching them into threaded holes in the base 302 (or vice versa).

At block 608, one or more compression elements may be inserted into the float mechanism 304. The compression elements may be, for example, springs 310a, 310b or compression foam and may be compression elements as described above in connection with FIGS. 3-5. In some cases, the compression elements may be integral with the float mechanism and therefore block 608 may be omitted. In some embodiments, the compression elements may be adjustable to change a degree to which the compression elements compress (and therefore change the amount of force absorbed by the compression elements). Optionally, at block 608 or later a technician may adjust the compression elements so that the amount of impact force and/or sustained force exerted on the optical transmitter/receiver 504 and/or lid 102 of the cassette falls within acceptable predetermined limits.

The float mechanism 304 may include an actuator, and accordingly at block 610 suitable actuation supply lines may be connected to the float mechanism 304. For example, one or more pneumatic fluid supply lines may be connected to pneumatic connectors 312a, 312b that are attached to the float mechanism 304. In embodiments using a different actuation power source, other appropriate types of supply lines (e.g., electrical wiring, hydraulic tubing, etc.) may be attached. The actuation supply lines may in turn be attached to a suitable actuation source, such as a compressor, pump, electrical source, etc.

At block 612, the probe support (proximal) 306 may be attached to the float mechanism 304. The probe support (proximal) 306 may be a probe support (proximal) 306 as described above in connection with FIGS. 3-5. The probe support (proximal) 306 may be attached to one of the sliding surfaces of the float mechanism 304 using fasteners and/or mating surfaces. For instance, one or more fasteners may be passed through through-holes in the probe support (proximal) 306 and into corresponding threaded holes in the float mechanism 304 (or vice versa).

At block 614, the probe-support (distal) 308 may be attached to the probe support (proximal) 306. The probe-support (distal) 308 may be a probe-support (distal) 308 as described above in connection with FIGS. 3-5. The probe-support (distal) 308 may be attached at one or more locations on the probe support (proximal) 306 using fasteners and/or mating surfaces. For instance, one or more fasteners may be passed through through-holes in the probe-support (distal) 308 and into corresponding threaded holes in the probe support (proximal) 306 (or vice versa). In some embodiments, the distal probe support arm 512 of the probe-support (distal) 308 may be fitted onto a proximal probe support arm 510 of the probe support (proximal) 306 to provide additional support in the vertical (in this example) direction. If vertical calibration adjusters 514 and/or horizontal calibration adjusters 516 are used, the adjusters may be affixed to the probe-support (distal) 308 and/or probe support (proximal) 306 at this block.

The sensor probe 506 may be connected to the probe-support (distal) 308 at block 616. In some embodiments where the sensor probe 506 includes a fiber optic cable 314, the fiber optic cable 314 may be attached to an electro-optical module for control and/or signal processing. The sensor probe 506 may be a sensor probe 506 as described above in connection with FIGS. 3-5 and may optionally be nested into corresponding indentations or mating elements in the probe-support (distal) 308. At block 618, a probe securer 508 may be affixed to the probe-support (distal) 308 to secure the sensor probe 506 in place.

At this stage, the sensor elevator may now be assembled and ready for adjustment. Accordingly, at block 620, a test cassette may optionally be provided at a predetermined location or configuration with respect to the sensor elevator and/or optical transmitter/receiver 504 (see FIG. 4). The actuator of the float mechanism 304 of the sensor elevator may be actuated to move the slides of the float mechanism 304 with respect to each other, e.g. until a maximum or minimum actuation distance is achieved. Alternatively, the actuator need not be moved to its full maximum or minimum extent, but may be moved to a predetermined stopping point. At the minimum/maximum actuation distance, the optical transmitter/receiver 504 should preferably be in a predetermined desired position with respect to the lid 102 and/or the oxygen sensor 402. The predetermined desired position may be a position where the optical transmitter/receiver 504 and lid 102 are a predetermined distance away from each other, where the optical transmitter/receiver 504 and lid 102 are just touching, or where the optical transmitter/receiver 504 and lid 102 are in contact with each other such that the compression elements of the float mechanism 304 are compressed. In the latter case, the predetermined desired position may be a position such that a contact and/or sustained force between the lid 102 and optical transmitter/receiver 504 is less than or equal to a predetermined maximum threshold amount.

Optionally, at block 622 the calibration adjusters may be manipulated so that the optical transmitter/receiver 504 and the lid 102 are moved to the predetermined desired position when the float mechanism 304 is at the maximum/minimum/predetermined actuation distance. Because of the presence of the compression elements in the float mechanism, the calibration of the position of the float mechanism with respect to the cassette is more forgiving, and the float mechanism 304 is able to better accommodate changes in the vertical position of the lid 102 if the cassette is not delivered to exactly the same position each time.

Once the sensor elevator is suitably calibrated, the testing cassette may be removed. At block 624, the sensor elevator may be used to measure the environment in cassettes that have been provided for analysis. Accordingly, a cassette may be provided for testing at block 624 by delivering the cassette to a predetermined location or configuration with respect to the sensor elevator (see FIG. 4). In both blocks 620 and block 624, the cassette may be delivered to the predetermined location by any suitable means, such as being moved and/or held in place by a robotic arm configured to manipulate the cassette during an analysis process.

Subsequently, at block 626 the float mechanism 304 may be actuated to move the float mechanism 304 to the minimum/maximum/predetermined distance as discussed above. If the optical transmitter/receiver 504 makes contact with the lid 102 at this stage, the impact and/or sustained forces are lessened by the action of the compression elements in the float mechanism 304.

In block 628, the sensor probe 506 may be used to perform measurements of the environment inside the cassette. For example a temperature sensor 502 may measure the temperature inside the cassette, and/or the optical transmitter/receiver 504 may transmit a beam of light onto the oxygen sensor 402 and receive the fluorescence that results. The fluorescence signal may be interpreted by an electro-optical module and provided to an analysis computer for processing or storage.

At block 628, after the measurements have been completed the float mechanism may be de-actuated or actuated in a reverse direction to move the optical transmitter/receiver 504 away from the lid 102. The cassette can then be safely removed from the vicinity of the sensor elevator at block 632 (e.g., using the robotic arm discussed above). At decision block 634, it may be determined if more cassettes remain to be tested. If so, then the method may revert to block 624 as the next cassette is moved into position. If not, then the method may proceed to done block 636 and complete.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

Some embodiments may be described using the expression “affixed,” “fastened,” “coupled” and “connected” along with their derivatives. These terms are not necessarily intended as synonyms for each other. For example, some embodiments may be described using the terms “affixed,” “fastened,” “connected” and/or “coupled” to indicate that two or more elements are in direct physical or electrical contact with each other. The terms “affixed,” “fastened,” and/or “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. An apparatus comprising:
a sensor probe; and
a sensor elevator configured to move the sensor toward a surface of a lid of a cassette to measure a property of an environment within the cassette, wherein the sensor elevator comprises a float mechanism configured to reduce or eliminate a sustained or impact force of the sensor on the lid of the cassette.

2. The apparatus of claim 1, wherein the sensor probe comprises one or more of an oxygen sensor probe, a temperature sensor probe, or a carbon dioxide sensor probe.

3. The apparatus of claim 1, wherein the sensor probe performs one or more of emitting light or receiving a fluorescence signal.

4. The apparatus of claim 1, wherein the float mechanism comprises a linear slide and one or more springs.

5. The apparatus of claim 1, wherein the float mechanism comprises foam cushioning.

6. The apparatus of claim 1, wherein the sensor elevator comprises a pneumatic actuator for moving the sensor probe toward the surface of the lid of the cassette.

7. The apparatus of claim 1, wherein the sensor elevator assembly comprises a base affixed to the float mechanism and configured to support the float mechanism at a predetermined location with respect to the cassette.

8. The apparatus of claim 1, wherein the sensor elevator assembly comprises a probe support to which the sensor probe is affixed, the probe support comprising a proximal member and a distal member.

9. The apparatus of claim 8, wherein the sensor elevator assembly comprises an adjuster configured to adjust at least one of the horizontal or vertical positioning of the distal member of the probe support with respect to the proximal member of the probe support.

10. The apparatus of claim 8, wherein:
the distal member of the probe support is T-shaped and comprises a distal probe support arm, and
the proximal member of the probe support is L-shaped and comprises a proximal probe support arm configured to interface with and support the distal probe support arm.

11. A method of deploying the apparatus of claim 1, comprising:
moving the cassette to a predefined measurement location with respect to the sensor elevator;
moving the sensor elevator to place the sensor probe into a predetermined configuration with respect to the cassette; and
measuring the property of the environment within the cassette using the sensor probe.

12. The method of claim 11, wherein moving the sensor elevator comprises moving the sensor probe into contact with the cassette so that the float mechanism compresses.

13. The method of claim 11, wherein measuring the property of the environment within the cassette comprises measuring an oxygen concentration, a temperature sensor probe, or a carbon dioxide concentration.

14. The method of claim 11, further comprising transmitting a control signal configured to cause the sensor probe to perform one or more of emitting light or receiving a fluorescence signal.

15. The method of claim 11, further comprising attaching a pneumatic line to a pneumatic actuator of the sensor elevator, and transmitting a control signal configured to cause the pneumatic actuator to move a predefined distance.

16. The method of claim 11, further comprising fixing a base to the float mechanism, the base configured to support the float mechanism at a predetermined location with respect to the cassette.

17. The method of claim 11, further comprising affixing the sensor probe to the sensor elevator.

18. The method of claim 17, wherein the sensor probe is fixed to a probe support that comprises a proximal member and a distal member.

19. The method of claim 18, wherein:
the distal member of the probe support is T-shaped and comprises a distal probe support arm, and
the proximal member of the probe support is L-shaped and comprises a proximal probe support arm configured to interface with and support the distal probe support arm; and further comprising assembling the distal member on the proximal member by mounting the distal member arm on the proximal member arm and fastening the proximal member arm and distal member arm together.

20. The method of claim 18, further comprising adjusting an adjuster to change at least one of the horizontal or vertical positioning of the distal member of the probe support with respect to the proximal member of the probe support.
